(19) United States
(12) Patent Application Publication
(10) Pub. No.: US 2012/0301541 A1
(43) Pub. Date: Nov. 29, 2012

(54) COMPRESSED CORE FOR PHARMACEUTICAL COMPOSITION

(76) Inventors: Elina HARONSKY, Rosh Ha'ain (IL); Dafna Arieli, Kadima (IL)

(21) Appl. No.: 13/479,930

(22) Filed: May 24, 2012

Related U.S. Application Data
(60) Provisional application No. 61/489,511, filed on May 24, 2011.

(51) Int. Cl. A61K 9/00; A61K 47/38; A61J 3/00; A61K 9/48; A61K 9/14; B05D 5/00; A61K 47/12; A61K 31/4439

(52) U.S. Cl. 424/452; 514/784; 514/781; 424/400; 514/338; 424/490; 427/2.21

(57) ABSTRACT

A compressed core for a pharmaceutical dosage form comprising a mixture of (a) at least one pharmaceutically acceptable organic acid, and (b) at least one pharmaceutically acceptable excipient is described. Such compressed core is useful for the preparation of pharmaceutical compositions containing a drug in which dissolution of the drug is favored in acidic environments. Also described are pharmaceutical compositions comprising such compressed core.

### CROSS REFERENCE TO RELATED APPLICATIONS

This patent application claims the benefit of U.S. Provisional Patent Application No. 61/489,511 filed May 24, 2011, the disclosure of which provisional application is herein incorporated by reference.

### FIELD OF THE INVENTION

The present invention relates to compressed cores which can be used for pharmaceutical compositions and dosage forms. The compressed cores of the present invention contain an organic acid, and are particularly useful for the preparation of pharmaceutical compositions containing a drug in which dissolution of the drug is favoured in acidic environments.

### BACKGROUND OF THE INVENTION

It is known that certain drugs, in particular weakly basic drugs and their salts, demonstrate solubilities that are pH-dependent. In standard matrix formulations, such drugs show a decreased release from the matrix once the formulation enters the higher pH environment of the gastrointestinal tract. The result of this is an unacceptably low, and potentially incomplete, release of the drug from the formulation.

Dabigatran, which has the IUPAC name: 3({2-[(4-carbamimidoylphenylamino)methyl]-1-methyl-1H-benzimidazole-5-carbonyl}-pyridin-2-yl-amino)propionic acid, and having the formula:

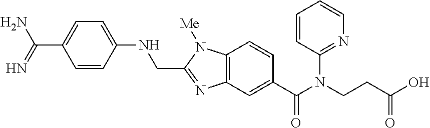

is an example of a drug having such a pH-dependent release profile. Dabigatran in the form of its prodrug, dabigatran etexilate, having the formula:

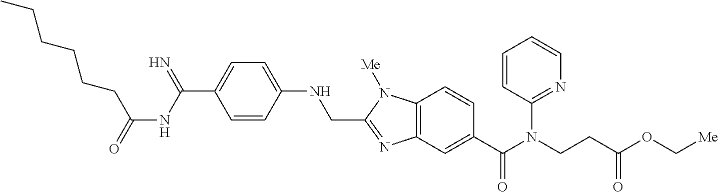

is an orally administered benzamidine thrombin inhibitor and has activity as an anticoagulant. Dabigatran etexilate (3-[(2-{4-(hexyloxycarbonylaminoiminomethyl)phenylamino]methyl}-1-methyl-1H-benzimidazole-5-carbonyl)pyridine-2-yl-amino]propionate) has use for the prevention of thrombosis, particularly for post-operative deep vein thrombosis, such as in, e.g., hip and knee replacement surgery, and also for the prevention or reduction of risk of stroke and systemic embolism, particularly in patients with non-valvular atrial fibrillation.

Dabigatran is described in U.S. Pat. No. 6,087,380. US 2010/0087488, US 2006/0247278 and US 2009/0042948 disclose various salts of dabigatran etexilate.

US 2005/0234104, US 2006/0276513, US 2008/0119523 and US 2010/0144796 describe various crystalline forms of dabigatran etexilate and its salts.

US 2005/0107438 describes dabigatran etexilate formulations in a dispersed form in an encapsulated lipophilic, pharmaceutically acceptable carrier system, which are said to provide oral formulations that are chemically and physically stable and have good bioavailability.

It is known that the solubility of weakly basic drugs, such as dabigatran and dabigatran etexilate, may be increased by the provision of an acidic environment. Hence, the provision of an acidic microenvironment at the intended site of drug release can increase the release rate from the dosage from.

For example, US 2005/0038077 describes a matrix tablet comprising dabigatran etexilate or a pharmaceutically acceptable salt thereof and one or more pharmaceutically acceptable organic acids and a pharmaceutically acceptable excipient or filler.

US 2003/0181488 describes oral formulations of dabigatran etexilate, which purport to provide pH-independent bioavailability of the active agent. The formulations contain a pharmaceutically acceptable organic acid having a water solubility of more than 1 g/250 ml at 20° C. The dosage forms are multiparticulate compositions containing pellets prepared by coating tartaric acid crystals of a specific particle size with a solution of tartaric acid dissolved in gum arabic. The coated crystals are sprinkled with powdered tartaric acid prior to screening to a specific size.

The disclosed formulation has disadvantages in particular because the process for its preparation is laborious as it requires several screening steps in order to achieve consistently sized particles for the encapsulated dosage form. Moreover, the multiple screening steps result in wastage of the starting materials and active substance, since the unsuitably sized particles at various stages of the process are discarded. Furthermore, the core preparation requires tartaric acid to be added in three different physical forms.

There is a continuing need to provide new and improved dosage forms of drugs having pH dependent solubilities, such as weakly basic drugs and their salts, including dabigatran. There is a further need to provide simplified and more cost effective processes for the preparation of the dosages forms of such drugs. The present invention addresses this need.

### SUMMARY OF THE INVENTION

In one aspect, the present invention provides a compressed core for a pharmaceutical dosage form comprising a mixture of (a) at least one pharmaceutically acceptable organic acid, and (b) at least one pharmaceutically acceptable excipient, wherein the pharmaceutically acceptable organic acid is present in an amount of about 50-95% by weight of the core. The core can be used as a component of a multilayer pharmaceutical composition containing a drug having pH dependent solubility. In particular, upon dissolution of the pharmaceutical composition, the core provides an acidic microenvironment in order to facilitate the dissolution of the drug from the pharmaceutical composition.

In a second aspect, the invention provides a process for the preparation of the compressed core comprising:
(i) admixing the pharmaceutically acceptable acid with the at least one pharmaceutically acceptable excipient to form a mixture, and
(ii) direct compression of the mixture.

In a third aspect of the present invention, there is provided a pharmaceutical composition comprising the compressed core wherein the core is coated with a drug layer comprising a drug having a pH dependent solubility profile, wherein the solubility is greater at acidic pH (i.e. pH<7), and at least one pharmaceutically acceptable excipient. The composition is preferably in the form of a mini tablet. The mini tablets can be used to prepare a final dosage form, e.g. by encapsulation.

In a fourth aspect, the present invention provides a process for preparing the pharmaceutical composition comprising the compressed core, wherein the process comprises:
(i) preparing a compressed core by the above process,
(ii) optionally applying a sub-coat layer over the compressed core,
(iii) applying a drug layer over the compressed core or sub-coated compressed core, and
(iv) optionally applying a protective top coat, an extended release coat or a delayed release coat over the drug layer.

### BRIEF DESCRIPTION OF THE DRAWINGS

FIG. 1A is a diagrammatic representation of a compressed core C in accordance with one embodiment of the invention

FIG. 1B shows a cross section through the compressed core of FIG. 1A

FIG. 2 is an enlarged photograph showing a capsule filled with subcoated cores according to an embodiment of the present invention (right) prepared according to Example 3, compared with capsules filled with pellets, such those used in the marketed Pradaxa® capsules (left).

FIG. 3 is an enlarged photograph showing sub-coated cores according to the present invention (left) prepared according to Example 3, compared with pellets such as those used in Pradaxa® (right).

### DETAILED DESCRIPTION OF THE INVENTION

As used herein, unless otherwise indicated, the term “drug having a pH dependent solubility” refers to a drug that has increased solubility when present in acidic environment (i.e. pH<7). Typically, the drug has a pKa in the range of from about 7 to about 14, preferably the pKa is greater than 7 and less than 12, more preferably the pKa is greater than 7 and less than 10.

As used herein, unless indicated otherwise, percentages refer to a weight percent. Weight percentages given in relation to the dosage form excludes the weight of any capsule shell.

As used herein, unless otherwise indicated, references to dabigatran includes references to enantiomers or prodrugs of dabigatran, such as dabigatran etexilate, as well as pharmaceutically salts (preferably mesylate, hydrochloride, maleate, tartrate, salicylate, citrate and malate salts, and particularly the mesylate salt), as well as solvates and hydrates of dabigatran, its enantiomers or prodrugs. The preferred form of dabigatran for any embodiment of the present invention is dabigatran etexilate, preferably in the form of its mesylate salt.

In a first aspect, the present invention provides a compressed core for a pharmaceutical dosage form comprising a mixture of (a) at least one pharmaceutically acceptable organic acid, and (b) at least one pharmaceutically acceptable excipient, wherein the pharmaceutically acceptable organic acid is present in an amount of about 50-95% by weight of the core.

The compressed core can be used in the preparation of pharmaceutical dosage forms of drugs that have a pH dependent solubility, in particular, drugs having a solubilities that are enhanced in acid conditions. The compressed core contains a high concentration of the pharmaceutically acceptable organic acid that on one hand provides an effective acid microenviroment, whilst enabling the resulting dosage form to maintain a compact size, which is desirable for patient compliance. Moreover, the compressed core can be easily and economically manufactured.

The compressed core of the invention described in any embodiment of the present invention contains the pharmaceutically acceptable organic acid in a high concentration, i.e. from about 50 to about 95 wt % of the core. Preferably, the pharmaceutically acceptable acid is present in the core in an amount of about 50 to about 90 wt % of the core, or about 50 to about 85% wt % of the core. Preferably, the pharmaceutically acceptable organic acid is present in an amount of greater than 50 wt % of the core. In particularly preferred embodiments, the pharmaceutically acceptable acid is present in an amount of about 60 to about 90 wt %, about 60 to about 85 wt %, about 70 to about 90 wt %, about 70 to about 85 w %, about 80 to about 85 wt %, about 80 to about 90 wt %, or about 85%, by weight of the core.

In any embodiment of the present invention, the pharmaceutically acceptable organic acid in the compressed core is one which upon administration is capable of producing an acid microenvironment in the gastrointestinal tract (i.e. pH<7, preferably pH<5.5, more preferably pH<5, or pH<4. The pharmaceutically acceptable organic acid preferably has a pKₐ of at least about 2, preferably wherein the pharmaceutically acceptable organic acid has a pKₐ of about 5.4 or less, preferably about 4 or less. The pharmaceutically acceptable organic acid preferably has a pKₐ of at least about 2.5, preferably at least about 2.9. Particularly, the pharmaceutically acceptable organic acid has a pKₐ of about 2.9 to about 5.4.

In any embodiment of the present invention, the pharmaceutically acceptable organic acid in the core has an aqueous solubility at 20° C. of 4 grams/litre, particularly 6 grams/litre, and especially 10 grams/litre.

Suitable pharmaceutically acceptable organic acids include, but are not limited to, fumaric acid, tartaric acid, citric acid, succinic acid, adipic acid, malic acid, maleic acid, lactic acid, or a mixture of one or more thereof. Of these, fumaric acid, tartaric acid, citric acid, and lactic acid are preferred. Tartaric acid, preferably L-tartaric acid is a preferred pharmaceutically acceptable acid in any embodiment of the present invention.

The present invention provides a core containing a pharmaceutically acceptable acid in the form of a compressed minitablet having a predetermined and uniform size. The cores of the present invention are preferably free of the any pharmaceutically active agent, and contain only the pharmaceutically acceptable acid and pharmaceutically acceptable excipients. Once coated with the drug, the uniformally sized core particles can be easily incorporated into a multiparticulate dosage form, e.g. by filling into a capsule or the like. At the same time, the cores of the present invention enable a high concentration of the pharmaceutically acceptable acid whilst being surprisingly mechanically stable. For example, typically, the compressed core has a friability of about 0.1% or less, preferably about 0.1%-0.02%, and more preferably about 0.1% to 0.01%. When the cores are used to manufacture a dosage form containing a drug having a pH dependent solubility, the cores dissolve and provide an acid microenvironment for the drug, thereby facilitating dissolution of the drug in the gastrointestinal tract.

Typically, pharmaceutically acceptable organic acids such as fumaric, tartaric, citric, succinic, adipic and malic acids are difficult to compress when incorporated at high concentration. Tartaric acid in particular is not considered to be a highly compressible material. Indeed, in US 2003/0181488, highly concentrated cores containing a pharmaceutically acceptable acid are prepared using crystals of the pure acid, to which a layer containing binder and further acid are applied as a solution by spray coating in a rotating pan. This method suffers from many disadvantages. In particular, at the outset, in order to achieve a narrow particle size range for the cores, the starting crystals of the acid are required to have a narrow particle size range. Moreover, several screening steps are required in order to maintain narrow particle size ranges during the processing of the cores into a dosage form.

The applicant has surprisingly found that pharmaceutically acceptable organic acids, and especially tartaric acid, can be compressed into tablets having small dimensions (i.e. so-called “minitablets”) by the inclusion of low concentrations of at least one pharmaceutically acceptable excipient selected from the group consisting of a filler (diluent) and binder, and optionally a lubricant, or a dissolution enhancer. Preferably, the at least one pharmaceutically acceptable excipient selected from the group consisting of a filler (diluent) and binder, and optionally a lubricant. In particular, the pharmaceutically acceptable acid can be in any form, and need not have a particular particle size range or particle size distribution. For example, the pharmaceutically acceptable acid can be in the form of a powder, or pellets. The pharmaceutically acceptable acid can be used directly without further steps (e.g. without a screening step). Preferably, the pharmaceutically acceptable excipient is a filler (diluent), or a mixture of a filler and a lubricant.

Suitable fillers (diluents) include microcrystalline cellulose (for example, Avicel PH102 having or PH101), lactose in its various forms (e.g. lactose USP, anhydrous or spray dried), sorbitol, dextrose, sucrose, mannitol, dibasic calcium phosphate, starch, and mixtures thereof, including mixtures of starch and lactose. Preferred are microcrystalline cellulose (such as Avicel PH102 having a nominal mean particle size of 100 microns), lactose in its various forms (e.g. lactose USP, anhydrous or spray dried), mannitol, dibasic calcium phosphate, starch, and mixtures thereof, including mixtures of starch and lactose. Of these, microcrystalline cellulose, mannitol, lactose, and starch, but particularly microcrystalline cellulose, lactose, and starch, are preferred. Microcrystalline cellulose is an especially preferred pharmaceutically acceptable excipient for use in the cores of the present invention.

Suitable binders include cellulose polymers, such as hydroxypropylmethyl cellulose, hydroxypropylcellulose, methylcellulose and hydroxyethyl cellulose, and polyvinylpyrrolidone and polyvinyl alcohol or mixtures thereof.

The core may optionally contain one or more lubricants. Examples of suitable lubricants include those selected from the group consisting of sodium stearyl fumarate, stearic acid, magnesium stearate, calcium stearate, zinc stearate, talc, glyceryl behenate, preferably sodium stearyl fumarate, magnesium stearate, calcium stearate and talc, and more preferably magnesium stearate or sodium stearyl fumarate. Magnesium stearate is a particularly preferred lubricant.

As noted above, the pharmaceutically acceptable organic acid is present in a high concentration in the core, i.e. from about 50 to about 95 wt % of the core. Preferably, the pharmaceutically acceptable acid is present in the core in an amount of about 50 to about 90 wt % of the core, or about 50 to about 85% wt % of the core. Preferably, the pharmaceutically acceptable organic acid is present in an amount of greater than 50 wt % of the core. In particularly preferred embodiments, the pharmaceutically acceptable acid is present in an amount of about 60 to about 90 wt %, about 60 to about 85 wt %, about 70 to about 90 wt %, about 70 to about 85 wt %, about 80 to about 85 wt %, about 80 to about 90 wt %, or about 85%, by weight of the core. Preferably the remainder is made up of the pharmaceutically acceptable excipient component (b). Thus, component (b) is preferably present in an amount of about 5-50%, about 10-50%, about 15-50%, about 10-40%, about 15-40%, about 10-30%, about 15-30%, about 20-30%, about 15-20%, about 10-20%, or about 15% by weight of the core.

In any of the above embodiments, a small quantity of lubricant may be added. For example, the lubricant may be present in the core in an amount of about 0.05 to about 2 wt %, preferably about 0.2 wt % to about 0.8 wt %, and more preferably about 0.3 to about 0.7 wt %, and particularly about 0.5 wt % (wt % are relative to the total weight of the core). A dissolution enhancer is generally included when a drug layer is applied. Therefore, when present, the dissolution enhancer is preferably present in an amount of 5-20% w/w of the core.

In any embodiment of the present invention, the weight ratio of the pharmaceutically acceptable acid (a) to the pharmaceutically acceptable excipient (b) in the core, is preferably about 1:1 to about 10:1, more preferably about 2:1, preferably about 4:1 to about 6:1. Thus, in a particularly preferred embodiment, the cores contain a pharmaceutically acceptable acid (a) in combination with a filler in a weight ratio of about 2:1, preferably about 4:1 to about 8:1. Optionally a lubricant may be included in a weight ratio of about 1:170 to about 1:200 relative to the total weight of components (a) and (b).

In particularly preferred embodiments, the compressed core consists essentially of a mixture of (a) in an amount of about 50-95 wt % of the pharmaceutically acceptable organic acid and (b) about 5-50 wt % (preferably about 10-20 wt %) of at least one pharmaceutically acceptable excipient. Preferably, in this embodiment, the pharmaceutically acceptable acid component (a) is typically present in an amount of about 60-95% by weight, and (b) is present in an amount of about 5-40% by weight of the core. More preferably, in this embodiment, the compressed core consists essentially of (a) in an amount of about 70-95% by weight, and (b) in an amount of about 5-30% by weight. Even more preferably, the compressed core consists essentially of (a) in an amount of about 80-90% by weight, and (b) in an amount of about 10-20% by weight. Preferably, in these embodiments, the pharmaceutically acceptable excipient consists essentially of a filler and optionally a lubricant, in concentrations (wt %) and weight ratios as discussed above. In these embodiments, the filler can be any of the filers as described above, although microcrystalline cellulose (e.g. Avicel PH 102) is particularly preferred. In the cores of these embodiments, a small quantity of lubricant as described above (but preferably magnesium stearate), may be added—preferably the lubricant is present in an amount of about 0.2 wt % to about 0.8 wt %, and more preferably about 0.3 to about 0.7 wt %, and particularly about 0.5 wt % (all wt % are relative to the total weight of the core).

The compressed cores of the present application as described in any of the above embodiments may be prepared by a process comprising direct compression of a mixture comprising components (a) and (b) and other optional components when present.

The compressed cores of the present invention may be further characterised by the absence of an effervescent couple. Such couples are familiar to those skilled on the art as being capable of generating a gas such as carbon dioxide in order to cause the dosage form to fizz and effervesce thereby rapidly releasing the drug from the dosage form.

Thus, a second aspect of the invention provides a process for the preparation of the compressed core of any of the embodiments described herein comprising:
(i) admixing the pharmaceutically acceptable acid as described in any of the above embodiments with the at least one pharmaceutically acceptable excipient as described in any of the above embodiments, to form a mixture, and
(ii) direct compression of the mixture.

The ingredients can be mixed or dry granulated prior to the compression step. The mixing or granulation is advantageously carried out without the use of any process solvent and/or soluble binder. For example, the ingredients for the core may be blended together using, e.g. a diffusion blender (optionally the lubricant, if present, is added after an initial blending step, followed by a further blending step after addition of the lubricant).

Typically, mixture for the direct compression can contain about 0.02 to about 4 wt % water (which may be present in the excipients), about 0.1 to about 4% water, and preferably about 0.5 to about 3% water.

The compression is carried out without the addition of a liquid or solvent, i.e. by direct compression. Typically, the mixture is compressed into tablets using a rotary tablet press. The so-formed compressed cores are typically in the form of minitablets which can be used directly as a component of a multilayer pharmaceutical composition or dosage form, i.e. without the need for a screening step.

The compressed cores of the present invention may be essentially cylindrical in shape, and have a diameter of the circular cross section of about 3 mm or less, or about 2 mm or less. Preferably, the cores have a diameter of at least about 1.6 mm. Preferably, the compressed core of any of embodiments described herein have a diameter range of about 1.6 to about 3 mm, about 1.6 to about 2.8 mm, particularly about 1.7 to about 2.5 mm and about 1.7 mm to about 2.3 mm, about 1.7 to about 2.1 mm, about 1.7 to about 2.0 mm, and particularly about 1.8 mm. The compressed core may also be spherical, or other shapes, depending on the die/punch used to carry out the compression. The spherical or other shaped compressed cores can have the same diameter ranges as set out above.

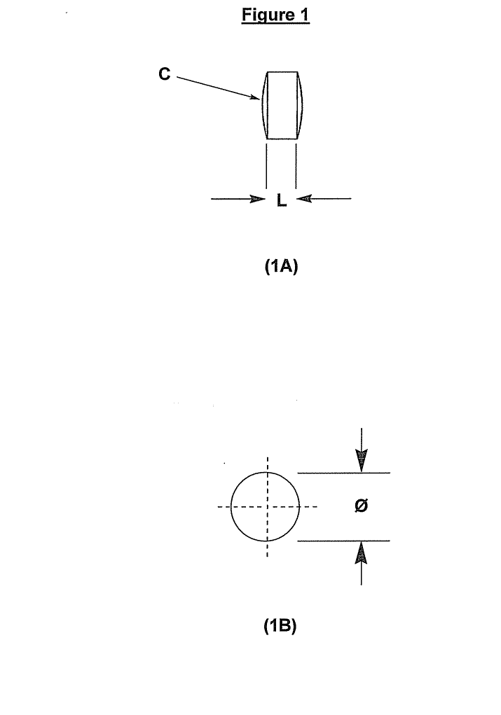

For example, in a preferred embodiment as shown in FIG. 1A, the compressed core C has a cylindrical shape, wherein the circular faces may be convex (shown) or may be flat. Typically, the compressed core has length L of about 1.2 mm to about 3 mm, preferably about 1.5 mm to about 2.5 mm and particularly about 2 mm. FIG. 1B shows a cross-section through the compressed core of FIG. 1A. The diameter Ø of the circular cross section of the compressed core can have range of about 1.6 to about 3 mm, about 1.6 to about 2.8 mm, particularly about 1.7 to about 2.5 mm and about 1.7 mm to about 2.3 mm, about 1.7 to about 2.1 mm, about 1.7 to about 2.0 mm, and particularly about 1.8 mm.

In preferred embodiments of the present invention, the compressed core comprises the pharmaceutically acceptable acid, particularly in an amount of 50 wt % to about 90 wt % relative to the weight of the core (preferably wherein the acid is tartaric acid, particularly L-tartaric acid), a filler (particularly microcrystalline cellulose, and especially Avicel PH102), and a lubricant (preferably magnesium stearate). Preferred concentrations of these components in the core are discussed in the preceding passages.

The cores having the described sizes are particularly suitable for the preparation of minitablets that can be encapsulated to produce the final dosage form, e.g. as a multiparticulate formulation, preferably in the form of encapsulated microtablets. In particular, the cores have a predetermined size and shape. Advantageously, the cores have a uniform size. As such, the use of multiple screening operations during processing of the cores and the dosage form in order to obtain suitably sized core particles having a narrow size distribution is avoided. Therefore, the present process is advantageous as it enables the production of uniformly sized cores, whilst avoiding the inevitable wastage from screening operations.

The cores of the present invention can be further processed into pharmaceutical dosage forms by providing a layer containing an active agent over the core, e.g. by coating methods. Thus, in a further aspect, the present invention provides a pharmaceutical composition comprising the compressed core as described in any of the above embodiments, wherein the core is coated with a drug layer comprising a drug having a pH dependent solubility profile, wherein the solubility is greater at acidic pH (i.e. pH<7), and at least one pharmaceutically acceptable excipient.

Preferably, in any embodiment of the present invention, the drug layer comprises an active agent in combination with at least one pharmaceutically acceptable excipient, preferably wherein the pharmaceutically acceptable excipient is selected from the group consisting of a binder, diluent, plasticizer and an anti-tacking (anti-adherant) agent, and mixtures thereof. Optionally, the drug layer comprises an active agent in combination with a binder, a plasticizer, an anti-tacking agent. The drug layer may comprise an active agent, in combination with a binder and an anti-tacking agent. Preferably, the drug layer comprises an active agent, in combination with a binder without an anti-tacking agent. More preferably, the drug layer doesn't comprise talc. Additionally the drug layer may include a dissolution enhancer.

The active agent can be present in a high concentration in the drug layer. Typically, the active agent can be present in a concentration of about 40 to about 90 wt %, about 50 to about 85 wt %, about 60 to about 80 wt %, and particularly about 70 to about 75 wt % relative to the weight of the drug layer. A high concentration of the active agent is desirable from the perspective of ensuring a smaller size of the dosage form.

The active agent in the drug layer is a drug that has a pH dependent solubility, in which the solubility of the drug is higher at lower pH. In particular the solubility increases at pH<7. Typically, the drug has a pKₐ in the range of from about 7 to about 14, preferably the pKa is greater than 7 and less than 12, more preferably the pKa is greater than 7 and less than 10. Such drugs are weak bases, and include: dabigatran, dabigatran prodrugs (preferably dabigatran etexilate) or pharmaceutically acceptable salts thereof (e.g. dabigatran etexilate mesylate), solvates or hydrates of dabigatran, dabigatran prodrugs and their pharmaceutically acceptable salts. The drug can also be selected from the group consisting of dipyridamole, aliskiren, fingolimod, and retigabin, and their pharmaceutically acceptable salts, as well as solvates and hydrates of these drugs or their pharmaceutically acceptable salts. In any of the embodiments of the invention, the drug is preferably dabigatran, dabigatran prodrugs (preferably dabigatran etexilate) or pharmaceutically acceptable salts thereof (e.g. dabigatran etexilate mesylate), solvates or hydrates of dabigatran. Dabigatran etexilate mesylate is a particularly preferred drug in the pharmaceutical compositions of any embodiment of the invention.

Suitable binders in the drug layer of the pharmaceutical composition of any embodiment of the present invention include any of the binders mentioned above for the core. For example, suitable binders include those selected from the group consisting of cellulosic polymers such as hydroxypropylmethyl cellulose, hydroxypropyl cellulose, hydroxyethyl cellulose, ethyl cellulose, gelatin, methyl cellulose, pregelatinized starch, acacia, alginic acid, sodium carboxymethyl cellulose gum arabic, polyvinyl pyrrolidone, polyvinyl alcohol, and copolymers of N-vinyl pyrrolidine and vinyl acetate or mixtures thereof. Hydroxypropylmethyl cellulose and hydroxypropyl cellulose (e.g. Klucel LF), or mixtures thereof, are particularly preferred binders for the drug layer, with hydroxypropyl cellulose being especially useful.

In any embodiment of the present invention, the binder in the drug layer can be present in a concentration of about 5 to about 30 wt %, about 5 to about 25 wt % and particularly about 10 to about 18 wt %, relative to the weight of the drug layer.

Preferably, in any embodiment of the present invention, the weight ratio of drug to binder in the drug layer is from about 10:1 to about 1:1, preferably about 8:1 to about 2:1 and more preferably about 6:1 to about 4:1.

Suitable plasticizers in the drug layer of the pharmaceutical composition of any embodiment of the present invention can include polyethylene glycol (particularly polyethylene glycol 400), triethyl citrate, tributyl citrate, glycerin, dibutyl sebacate, triacetin and diethylphthalate. Particularly preferred are polyethylene glycol, triethyl citrate, tributyl citrate, dibutyl sebacate, triacetin and diethyl phthalate, or mixtures thereof.

In any embodiment of the present invention, where present in the drug layer, the plasticizer may be present in the drug layer in a concentration of about 2 to about 25 wt %, about 5 to about 15 wt % or about 8 to about 12 wt % relative to the weight of the drug layer.

In any embodiment of the present invention, an anti-tacking agent (anti-adherant) may be included in the drug layer. The anti-tacking agent can include magnesium carbonate, titanium dioxide, microcrystalline cellulose, polyethylene glycol, colloidal silica, corn starch and talc, or mixtures thereof. Talc (especially extra fine talc) is a particularly preferred anti-tacking agent.

Where present in the drug layer, the anti-tacking agent can be employed in a concentration range of about 5 wt % to about 25 wt %, about 8 wt % to about 20 wt %, or about 10 wt % to about 18 wt % relative to the weight of the drug layer.

When present, the dissolution enhancer is preferably present in an amount of 5-20% w/w of the layer or region it is present in i.e. of the core, drug layer or sub-coating layer. Preferably the dissolution enhancer is a pore former contained in the drug layer, preferably such that the weight ratio of dissolution enhancer to drug is from about 1:20 to about 10:1. For example, in the case of a drug-layer containing 150 mg Dabigatran, the preferred amount of a pore-former is from about 3 mg to about 50 mg.

In a particularly preferred embodiment, the drug layer is composed of the active agent as described in any of the above embodiments (e.g. dabigatran, its prodrugs, or pharmaceutically acceptable salts, solvates and hydrates thereof, such as dabigatran etexilate mesylate), in combination with a binder as described above (e.g. a cellulose polymer such as the hydroxyalkyl celluloses including hydroxypropylmethyl cellulose, hydroxypropyl cellulose) and an anti-tacking agent (preferably talc). The concentrations of these components are as set out in the preceding passages.

The drug layer may be applied to the compressed cores as described in any of the embodiments herein by any coating procedure, including by fluid-bed coater, by pan-coating or by spray coating. Preferably, the drug layer and/or the subcoat layer are applied to the compressed cores by pan-coating. Pan-coating is much more simple, energy efficient and cheaper coating process. Typically, the ingredients for the drug layer are mixed together in, e.g. C₁₋₃ alcohols such as ethanol, isopropanol, or mixtures thereof, and optionally in combinations of the alcohol with purified water to form a coating solution, which can be applied by the above coating methods. Since the cores are of uniform size, there is no need for a screening step following the drug-layer coating step in order to obtain uniform particles.

In certain embodiments of the pharmaceutical compositions of the present invention, it may be preferable to include a subcoat layer between the core containing the pharmaceutically acceptable acid and the drug layer. The inclusion of a subcoat layer is particularly useful for providing a physical barrier to protect certain active agents, including dabigatran, from undesirable interactions with the acid in the core.

When present, the subcoat layer may comprise at least one pharmaceutically acceptable excipient selected from one or more of the group consisting of binder (preferably wherein the binder is a water-soluble polymer), anti-tacking agent, surfactant (emulsifier), dissolution enhancer and plasticizer. The subcoat layer preferably comprises at least one pharmaceutically acceptable excipient selected from one or more of the group consisting of binder (preferably wherein the binder is a water-soluble polymer), anti-tacking agent, surfactant (emulsifier), and plasticizer. Optionally, the subcoat layer does not comprise an anti-tacking agent. In particular, the subcoat layer doesn't comprise talc. Optionally, the sub-coat layer can include a further amount of a pharmaceutically acceptable organic acid such as those described above in the context of the core.

The binder in the subcoat layer may be selected from those binders listed above for the drug layer. Thus, suitable binders for the subcoat layer include cellulosic polymers such as hydroxypropylmethyl cellulose, hydroxypropyl cellulose, hydroxyethyl cellulose, ethyl cellulose, gelatin, methyl cellulose, pregelatinized starch, acacia, alginic acid, sodium carboxymethyl cellulose gum arabic, polyvinyl pyrrolidone, polyvinyl alcohol, and copolymers of N-vinyl pyrrolidine and vinyl acetate, or a mixture thereof. Of these, the cellulosic polymers, e.g. hydroxypropylmethyl cellulose, hydroxypropyl cellulose, hydroxyethyl cellulose, ethyl cellulose and methyl cellulose are preferred. Hydroxypropylmethyl cellulose (e.g. HPMC 2910), hydroxypropyl cellulose, hydroxyethyl cellulose and ethyl cellulose or mixtures thereof, are particularly preferred binders for the subcoat layer. Preferably the binders for the subcoat are hydroxypropylmethyl cellulose and ethyl cellulose or a combination thereof.

The binder is typically present in the subcoat layer in a concentration of about 20 to about 95 wt %, about 30 to about 90 wt %, or about 40 to about 90 wt %, relative to the weight of the subcoat layer.

Where present in the subcoat layer, the anti-tacking agent can be any of the anti-tacking agents employed in the drug layer. Thus, for example, the anti-tacking agent may include magnesium carbonate, titanium dioxide, microcrystalline cellulose, polyethylene glycol (particularly polyethylene glycol 6000), colloidal silica, corn starch and talc or mixtures thereof. Talc is a particularly preferred anti-tacking agent.

Where present in the subcoat layer, plasticizer can be any of the plasticizers employed in the drug layer. Examples of these include polyethylene glycol (particularly polyethylene glycol 400), triethyl citrate, tributyl citrate, glycerin, dibutyl sebacate, triacetin and diethylphthalate. Particularly preferred are polyethylene glycol, triethyl citrate, tributyl citrate, dibutyl sebacate, triacetin and diethyl phthalate, or mixtures thereof, and especially polyethylene glycol and dibutyl sebacate, or a combination thereof.

Typically, the plasticizer may be employed in the subcoat in a concentration of about 5 to about 30 wt %, about 5 to about 20 wt %, or about 8 to about 14 wt %, relative to the weight of the subcoat.

Where present in the subcoat, the surfactant or emulsifier is preferably selected from benzalkonium chloride, cetyl alcohol, polysorbate 80, sodium lauryl sulfate and sorbitan esters including sorbitan mono-palmitate or mixtures thereof, and particularly cetyl alcohol or sodium lauryl sulfate, or a combination thereof.

The surfactant may be employed in low concentrations, for example about 0.05 to about 6 wt %, typically about 0.1 to about 1 wt % or about 0.2 wt % to about 0.5 wt %.

An especially suitable ready-made subcoat in the form of Opadry clear (Colorcon), which contains hypromellose 15 cPS(HPMC 2910), ethyl cellulose 10 cPs, polyethylene glycol 400, dibutyl sebacate, cetyl alcohol and sodium lauryl sulfate.

A dissolution enhancer is generally included when a drug layer is applied. Therefore, when present, the dissolution enhancer is preferably present in an amount of 5-20% w/w of the sub-coating layer.

The subcoat layer may be applied in a similar manner to the drug layer. For example the ingredients for the subcoat layer can be mixed together in, e.g. C₁₋₃ alcohols such as ethanol, isopropanol, or mixtures thereof, and optionally in combinations of the alcohol with purified water, to form a coating solution, which can be applied by the various coating methods as discussed above for the drug layer (e.g. using fluid bed coater).

In any embodiment of the pharmaceutical compositions described herein, the drug layer may be provided with a further coating. This further coating may be a protective top coat, or a top coat that provides particular release properties, e.g. a extended-release coat or a delayed-release coat, as appropriate for the drug and dosage form.

The protective top coat can include a binder, an anti-tacking agent and a plasticizer.

Suitable binders, anti-tacking agents and plasticizers, include those described above for the drug layer or the subcoat layer. The binder can be any of those mentioned including the preferred agents described above in relation to the drug layer or subcoat layer. The binder may be present in the top coat in an amount of about 20 to about 60 wt %, about 30 to about 60 wt %, or about 40 to about 50 wt % relative to the weight of the top coat. The anti-tacking agent can be any of those mentioned including the preferred agents described above in relation to the drug layer or subcoat layer. The anti-tacking agent may be present in the top coat in an amount of about 20 to about 60 wt %, about 30 to about 60 wt %, or about 40 to about 50 wt % relative to the weight of the top coat. The plasticizer can be any of those mentioned including the preferred agents described above in relation to the drug layer or subcoat layer. The plasticizer may be present in the top coat in an amount of about 2 to about 40 wt %, about 5 to about 20 wt %, or about 8 to about 12 wt % relative to the weight of the top coat. Particularly preferred is a top coat comprising hydroxypropylmethyl cellulose (especially HPMC 2910), talc and polyethylene glycol (particularly PEG 400).

As to extended-release coating, this may comprise an extended-release polymer, a binder, and a plasticizer. The plasticizer component can be any of the plasticizers mentioned above for the drug layer or the subcoat, and thus includes polyethylene glycol (particularly polyethylene glycol 400), triethyl citrate, tributyl citrate, glycerin, dibutyl sebacate, triacetin and diethylphthalate. Particularly preferred are polyethylene glycol, triethyl citrate, tributyl citrate, dibutyl sebacate, triacetin and diethyl phthalate, or mixtures thereof, and especially triethyl citrate. Preferably, the plasticizer can be used in a concentration of about 2 to about 30 wt %, about 5 to about 20 wt %, or about 10 to about 18 wt %, relative to the weight of the extended release coating.

The binder component can be any of the binders mentioned above for the drug layer or the subcoat, and is preferably selected from the group consisting of cellulosic polymers such as hydroxypropylmethyl cellulose, hydroxypropyl cellulose, hydroxyethyl cellulose, ethyl cellulose, gelatin, methyl cellulose, pregelatinized starch, acacia, alginic acid, sodium carboxymethyl cellulose gum arabic, polyvinyl pyrrolidone, polyvinyl alcohol, and copolymers of N-vinyl pyrrolidine and vinyl acetate, or a mixture thereof. Cellulosic polymers, and preferably hydroxypropylmethyl cellulose, hydroxypropyl cellulose and hydroxyethyl cellulose are preferred. Hydroxypropylmethyl cellulose (e.g. HPMC 2910) is especially preferred. The binder is preferably present in a concentration of about 2 to about 30 wt %, preferably about 5 to about 25 wt %, and particularly about 10 to about 20 wt %, relative to the weight of the extended-release coating.

Typically the extended-release polymer can selected from the group consisting of ethyl cellulose (e.g. ethylcellulose having a viscosity of about 4 to about 10 cPs, preferably about 5 to about 9 cPs, and more preferably about 7 cPs), hydroxypropyl methylcellulose (HPMC), polyvinyl alcohol (PVA; vinyl alcohol polymer), polymethacrylates, ethyl acrylate-methyl methacrylate copolymers (such as Eudragit RS), hydroxypropyl cellulose (HPC) or a mixture thereof. Preferably, the extended-release polymer is ethylcellulose (such as ethylcellulose having a viscosity of about 4 to about 10 cPs, preferably about 5 to about 9 cPs, and more preferably about 7 cPs). The extended-release polymer can be present in a concentration of about 20 to about 85 wt %, about 40 to about 80 wt %, or about 55 to about 70 wt % relative to the weight of the extended release coating.

A suitable delayed release coating may comprise an enteric polymer, a plasticizer and an anti-tacking agent.

Suitable enteric polymers include methacrylate copolymers (e.g. Eudragit L30 D55—an anionic polymethacrylate), hydroxypropylmethyl cellulose phthalate, hydroxypropylmethyl cellulose acetate succinate and polyvinylacetate phthalate. The enteric polymer can be used in a concentration of from about 20 to about 85 wt %, about 40 to about 80 wt %, or about 55 to about 70 wt % relative to the weight of the delayed release coating.

Suitable anti-tacking agents can include magnesium carbonate, titanium dioxide, microcrystalline cellulose, polyethylene glycol, colloidal silica, corn starch and talc, or mixtures thereof, and preferably talc.

The plasticizer component of the delayed release coating can be any of the plasticizers mentioned above for the drug layer or the subcoat, and thus includes polyethylene glycol (particularly polyethylene glycol 400), triethyl citrate, tributyl citrate, glycerin, dibutyl sebacate, triacetin and diethylphthalate. Particularly preferred are polyethylene glycol, triethyl citrate, tributyl citrate, dibutyl sebacate, triacetin and diethyl phthalate, or mixtures thereof, and especially triethyl citrate. Preferably, the plasticizer can be used in a concentration of about 2 to about 30 wt %, about 5 to about 15 wt %, or about 7 to about 12 wt % relative to the weight of the delayed release coating.

The top coat, extended release coat and the delayed release coat can be applied by the coating procedures described above for the drug layer and the subcoat.

As used above, the term “dissolution enhancer” refers to any excipient that has the ability to function in such a manner. In particular, pore formers, osmotic agents, surfactants and disintergrants are included as suitable dissolution enhancers. Unless explicitly stated otherwise, the dissolution enhancer is present in an amount of from 5-20% w/w of the layer or region it is present in i.e. of the core, drug or su-coating layer.

Preferably, the dissolution enhancer is a pore former such as polyethylene glycol with molecular weight of 200-8000 g/mol, lactose or lactose monohydrate, mannitol, sodium chloride, triethyl citrate, low viscosity polyvinyl alcohol, dibasic calcium phosphate and talc. Alternatively, the dissolution enhancer may be a disintergrant such as crospovidone, croscarmellose sodium, low substituted hydroxypropyl cellulose and sodium starch glycolate in an amount of about 0.5-8%, 1-7%, preferably 2-5% by weight of the total composition.

The dissolution enhancer is preferably a pore former. The pore former is preferably a water soluble pharmaceutical excipient which is mixed with at least one polymeric film former (such as the binder discussed above) and optionally with additional component, which can be the drug (if in the drug layer) or excipient, to form a film. The pore former increases the porosity, and thereby the solubility of the resulted film. The preferred ratio between the pore-former and the film-former is from about 1:20 to about 10:1. Preferably the pore former is contained in the drug layer. When the drug layer comprises a pore former, the pore former is in an amount of about 5-20% w/w of the total drug-layer composition. For example, in the case of a drug-layer containing 150 mg Dabigatran, the preferred amount of a pore-former is from about 3 mg to about 50 mg.

According to a further aspect of the present invention, there is provided a process for preparing the pharmaceutical composition as described in any of the above embodiments, comprising
(i) preparing a compressed core as described above,
(ii) optionally applying a sub-coat layer over the compressed core,
(iii) applying a drug layer over the compressed core or sub-coated compressed core, and
(iv) optionally applying a protective top coat, an extended release coat or a delayed release coat over the drug layer.

The components of the compressed core, sub-coat layer, drug layer, top coat, extended release coat and delayed release are as discussed in any of the embodiments described above.

As the compressed cores can be made to a predetermined and uniform particle size, the cores are particularly suitable for the preparation of multiparticulate dosage forms of drugs having pH dependent solubility release as discussed above particularly in the form of capsules containing drug-coated minitablets. The compressed cores are particularly useful for preparing pharmaceutical compositions of a drug selected from the group consisting of dabigatran, dabigatran prodrugs (preferably dabigatran etexilate) or pharmaceutically acceptable salts thereof (e.g. dabigatran etexilate mesylate), as well as dipyridamole, aliskiren, fingolimod, and retigabin, and their pharmaceutically acceptable salts. In particular, these drugs are characterised by having a pH dependent solubility, i.e. increasing solubility with decreasing pH.

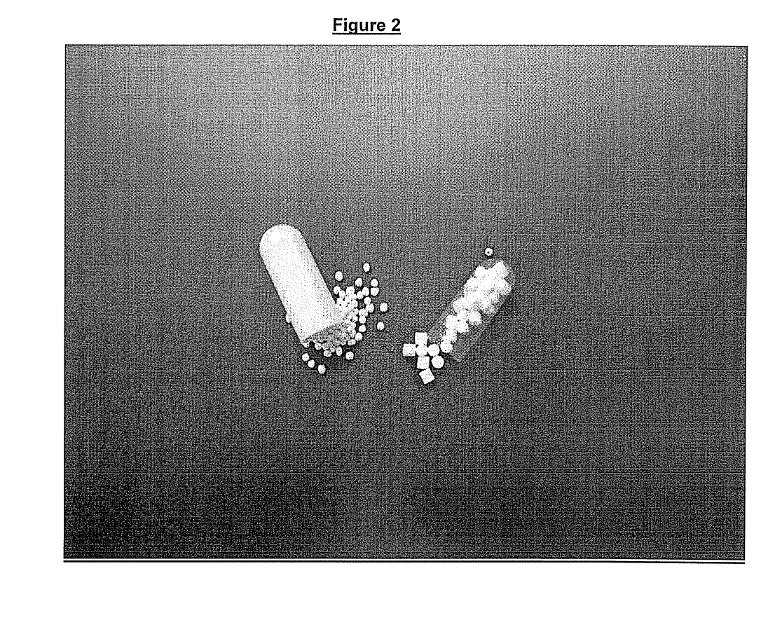

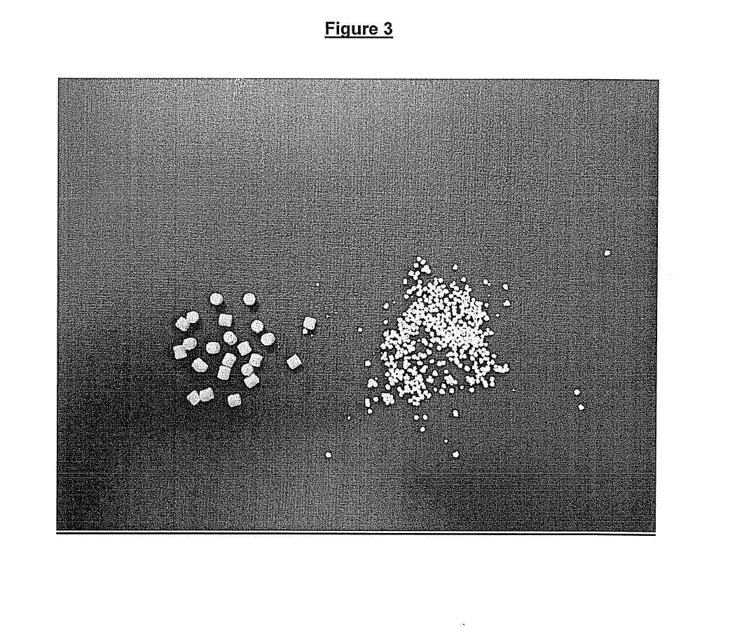

Thus, in a further aspect, the present invention provides a multiparticulate dosage form, comprising a plurality of coated cores as defined in any of the embodiments described above. The multiparticulate dosage form can be in the form of capsules filled with the coated cores. The coated cores are typically in the form of minitablets having an essentially cylindrical shape (e.g. similar to the compressed cores shown in FIGS. 1A and 1B). The circular surfaces at each end of the cylinder shape may be convex. The coated cores may have other shapes depending on shape of the compressed core as discussed above. For example, the coated cores can be spherical or other shapes. The circular cross section diameter and length of the coated cores will be slightly larger than the diameter Ø and length L of the cores (as shown in FIGS. 1A and 1B) due to the presence of the coating(s). Preferably, the coated cores have a circular cross section diameter of greater than about 1.6 mm or more, at least about 1.8 mm or more, preferably about 1.6 to about 4 mm, about 2 to about 4 mm, about 2 to about 3 mm, or about 2.4 to about 2.6 mm. Preferably the coated cores have a length of about 2.4 to about 4 mm more preferably, 2.6 to about 3.5 mm and most preferably 2.8. In the case of spherical or other shaped cores, the diameters correspond to the diameters ranges of the circular cross section of the cylindrical cores as set out above. The coated cores (e.g. minitablets) are typically larger in size compared with the approximately spherical pellets used in the formulation of dabigatran etexilate marketed under the name Pradaxa® (FIGS. 2 and 3), which is believed to be manufactured according to the rotating pan-coating process described in US 2003/0181488. In view of their larger size and excellent size uniformity, the tablets of the present invention are easier to fill into capsules for a final dosage form.

In particular, in accordance with a further aspect of the present invention, there is provided a process for preparing a pharmaceutical dosage form comprising filling the pharmaceutical composition according to any embodiment of the invention (e.g. the minitablets), or a plurality thereof, into a capsule, preferably wherein the capsule is a hard gelatin capsule or hydroxypropylmethyl cellulose capsule.

Moreover, the present invention provides the coated cores containing the drug and the acid in a concentrated form, which enables the cores to be filled into smaller capsules whilst retaining the dosage size, which reduces the problems associated with large dosage forms (e.g. difficulty in swallowing, and hence poor patient compliance).

The invention is illustrated by the following examples, which do not limit the scope of the invention. It will be appreciated that various modifications are within the spirit and scope of the invention.

### EXAMPLES

### Example 1

### Tartaric Acid Tablet Cores Containing Tartaric Acid Powder

L-tartaric acid and microcrystalline cellulose were combined into a blend using a diffusion blender for 5 min. The mixture obtained was then blended with magnesium stearate for additional 3 min. The final mixture was compressed into 1.8 mm tablets (i.e. cylindrical cores wherein the circular cross section is 1.8 mm in diameter) by a rotary tablet press. A batch size of 24,000 tablets was produced with good yield.

Table 1 summarizes the composition of the tablets of Example 1:

TABLE 1
Formulation of tablets of Example 1 by weight

| Component | mg/tab |
|---|---|
| Tartaric acid powder 75-300 μm | 6.37 |
| Microcrystalline cellulose (Avicel PH 102) | 1.09 |
| Magnesium stearate | 0.04 |
| Total weight | 7.50 |

### Example 2

### Tartaric Acid Tablet Cores Containing Tartaric Acid Pellets

Table 2 summarizes the composition of the tablets of Example 2, prepared in a procedure similar to the one described in Example 1:

TABLE 2
Formulation of tablets of Example 2 by weight

| Component | mg/tab |
|---|---|
| Tartaric acid pellets 400-600 μm | 6.37 |
| Microcrystalline cellulose (Avicel PH 102) | 1.09 |
| Magnesium stearate | 0.04 |
| Total weight | 7.50 |

### Example 3

### Tartaric Acid Tablet Cores Coated with Hypromellose Sub Coat

The tartaric acid cores prepared according to example 1 were coated by a 10% w/w isolating layer, its composition is described in Table 3.

The coating was carried out using a small scale pan-coater (7000 tablets/batch)

TABLE 3
Formulation of tablets of Example 3 by weight

| | |
|---|---|
| Component | mg/tab |

Core

| | |
|---|---|
| Tartaric acid cores (Example 1) | 7.50 |

Coating

| | |
|---|---|
| Hypromellose 6 cPs (HPMC 2910) | 0.364 |
| Talc extra fine | 0.364 |
| Polyethylene Glycol 400 | 0.082 |
| Ethanol 95% (*) | |
| Total weight | 8.31 |

(*) Removed during process

FIG. 2 (capsule on the right) and FIG. 3 (capsule on the left) show a comparison of the sub-coated cores prepared according to this process, with the marketed Pradaxa® capsules (left in FIG. 2 and right in FIG. 3)

### Example 4

### Tartaric Acid Tablet Cores Coated with Hypromellose Sub Coat

The tartaric acid cores prepared according to example 2 were coated by a 10% w/w isolating layer, its composition is described in Table 4.

The coating was carried out using a small scale fluid-bed coater (7000 tablets/batch)

TABLE 4
Formulation of tablets of Example 4 by weight

| | |
|---|---|
| Component | mg/tab |

Core

| | |
|---|---|
| Tartaric acid cores (Example 2) | 7.50 |

Coating-Opadry Clear 21F29126

| | |
|---|---|
| Hypromellose 15 cPS (HPMC 2910) | 0.575 |
| Ethyl cellulose 10 cPs | 0.146 |
| Polyethylene Glycol 400 | 0.085 |
| Dibutyl Sebacate | 0.002 |
| Cetyl Alcohol | 0.002 |
| Sodium Lauryl Sulfate | 0.001 |
| Ethanol 95% (*) | |
| Total weight | 8.31 |

(*) Removed during process

### Example 5

### Tartaric Acid Tablet Cores Coated with Hypromellose Sub Coat and a Drug Layer of Dabigatran Ethexylate

The tartaric acid cores prepared according to example 3 are coated with a 55% w/w drug layer, its composition is described in Table 5.

The coating is carried out using a medium-scale pan-coater (70000 tablets/batch)

TABLE 5
Formulation of tablets of Example 5 by weight

| | |
|---|---|
| Component | mg/tab |

Core + Subcoat

| | |
|---|---|
| Tartaric acid cores (Example 1) | 7.50 |
| Subcoat (Example 3) | 0.81 |

Coating-drug layer

| | |
|---|---|
| Dabigatran etexilate mesylate (d(0.9) LT 50 um) (*) | 7.21 |
| Hydroxypropyl cellulose (Klucel LF) | 1.45 |
| Talc extra fine | 1.43 |
| Isopropyl Alcohol (**) | |
| Total weight | 18.40 |

(*) 24 tablets contain 173.0 mg Dabigatran etexilate mesylate, which are equivalent to 150 mg Dabigatran etexilate
(**) Removed during process

### Example 5a

### Tartaric Acid Tablet Cores Coated with Hypromellose Sub Coat and a Drug Layer of Dabiqatran Ethexylate without Talc

The tartaric acid cores prepared according to example 2 were coated by an isolating layer according to example 3 and further coated with a drug layer, its composition is described in Table 5a.

The coating was carried out using a medium-scale pan-coater (70000 tablets/batch)

TABLE 5a
Formulation of tablets of Example 5a by weight

| | |
|---|---|
| Component | mg/tab |

Core + Subcoat

| | |
|---|---|
| Tartaric acid cores (Example 2) | 7.50 |
| Subcoat (Example 3) | 0.81 |

Coating-drug layer

| | |
|---|---|
| Dabigatran etexilate mesylate (d(0.9) LT 50 um) (*) | 7.21 |
| Hydroxypropyl cellulose (Klucel LF) | 1.45 |
| Isopropyl Alcohol (**) | |
| Total weight | 16.97 |

(*) 24 tablets contain 173.0 mg Dabigatran etexilate mesylate, which are equivalent to 150 mg Dabigatran etexilate
(**) Removed during process

### Example 6

### Tartaric Acid Tablet Cores Coated with Hypromellose Sub Coat, a Drug Layer of Dabigatran Etexilate and a Top-Coat

The tartaric acid cores prepared according to example 5 are coated with an 8% w/w top-coat; its composition is described in Table 6.

The coating is carried out using a medium-scale pan-coater (70000 tablets/batch).

TABLE 6
Formulation of tablets of Example 6 by weight

| | |
|---|---|
| Component | mg/tab |

Core + Subcoat + Drug Layer

| | |
|---|---|
| Tartaric acid cores (Example 1) | 7.50 |
| Subcoat (Example 3) | 0.81 |
| Drug Layer (Example 5) | 10.09 |

Top-Coating layer

| | |
|---|---|
| Hypromellose 6 cPs (HPMC 2910) | 0.72 |
| Talc extra fine | 0.72 |
| Polyethylene Glycol 400 | 0.16 |
| Ethanol 95% (*) | |
| Total weight | 20.00 |

(*) Removed during process

### Example 7

### Tartaric Acid Tablet Cores Coated with Hypromellose Sub Coat, a Drug Layer of Dabigatran Ethexylate and an Extended Release Coat

The tartaric acid cores prepared according to example 5 are coated with a 17% w/w extended release layer; its composition is described in Table 7.

The coating is carried out using a medium-scale pan-coater (70000 tablets/batch).

TABLE 7
Formulation of tablets of Example 7 by weight

| | |
|---|---|
| Component | mg/tab |

Core + Subcoat + Drug Layer

| | |
|---|---|
| Tartaric acid cores (Example 1) | 7.50 |
| Subcoat (Example 3) | 0.81 |
| Drug Layer (Example 5) | 10.09 |

Coating- extended release layer

| | |
|---|---|
| Ethylcellulose 7 cPs | 2.58 |
| Hypromellose 6 cPs (HPMC 2910) | 0.56 |
| Triethyl Citrate | 0.56 |
| Ethanol 95% (*) | |
| Isopropyl Alcohol (*) | |
| Purified water (*) | |
| Total weight | 22.10 |

(*) Removed during process

### Example 8

### Tartaric Acid Tablet Cores Coated with Hypromellose Sub Coat, a Drug Layer of Dabigatran Etexilate and an Delayed Release Coat

The tartaric acid cores prepared according to example 5 are coated with a 17% w/w delayed release layer; its composition is described in Table 8.

The coating is carried out using a medium-scale pan-coater (70000 tablets/batch).

TABLE 8
Formulation of tablets of Example 8 by weight

| | |
|---|---|
| Component | mg/tab |

Core + Subcoat + Drug Layer

| | |
|---|---|
| Tartaric acid cores (Example 1) | 7.50 |
| Subcoat (Example 3) | 0.81 |
| Drug Layer (Example 5) | 10.09 |

Coating- delayed release layer

| | |
|---|---|
| Eudragit L-30 D55 (Anionic | 2.32 |
| polymethacrylate) | |
| Triethyl Citrate | 0.33 |
| Talc Extra fine | 1.05 |
| Purified water * | |
| Total weight | 22.10 |

* Removed during process

### Example 9

### Encapsulated Tartaric Acid Tablet Cores Coated with Hypromellose Sub Coat and a Drug Layer of Dabigatran Etexilate

The tartaric acid cores prepared according to example 5 are encapsulated into hard-gelatin or hydroxypropylmethyl cellulose capsules using conventional encapsulation machine equipped with an appropriate filling disk according to Table 9:

TABLE 9
Final capsules of current invention

| | | | # of Coated |
|---|---|---|---|
| Strength (DE) | Fill weight [mg] | Capsule size | Tablets/Cap |
| 75 mg | 220.8 | 3 | 12 |
| 150 mg | 441.6 | 1 | 24 |

### Example 10

### Reference Example (Encapsulated Commercial Pellets)

The capsules described in Example 9 are expected to be bio-equivalent to the commercial drug-layer containing pellets, Pradaxa®, which can be described by Table 10:

TABLE 10
Final capsules of PRADAXA ®

| | | | # of Coated |
|---|---|---|---|
| Strength (DE) | Fill weight [mg] | Capsule size | Pallets/Cap |
| 75 mg | 215 | 2 | Ca 250 |
| 150 mg | 430 | 0 | Ca 500 |

### Example 11

### Suitability of Tartaric Acid for Compression

The Compressibility Index and Hausner Ratio are measures of the propensity of a powder to be compressed.

The compressibility index and Hausner ratio may be calculated (USP 35-NF 30, General Chapters: <1174> POWDER FLOW:) using measured values for bulk density (P_{bulk}) and tapped density (P_{tapped}) as follows:

Compressibility Index=100×[(P_{tapped}−P_{bulk})/P_{tapped}]

Hausner Ratio=(P_{tapped}/P_{bulk})

Alternatively, the compressibility index and Hausner ratio may be calculated by measure (1) the unsettled apparent volume, Vₒ, and (2) the final tapped volume, V_{f}, of the powder after tapping the material until no further volume changes occur.

The compressibility index and the Hausner ratio are calculated as follows:

Compressibility Index=100×[(Vₒ−V_{f})/Vₒ]

Hausner Ratio=(Vₒ/V_{f})

By measuring bulk density and tapped density it has been shown that it was not trivial to compress both the tartaric acid pellets and the tartaric acid powder: 85% tartaric acid powder/15% microcrystalline cellulose (Avicel PH102)

| | 85% tartaric acid | 85% tartaric acid |
|---|---|---|
| | powder/15% | pellets/15% |
| | microcrystalline cellulose | microcrystalline cellulose |
| | (Avicel PH102) R-07506 | (Avicel PH102) K-45625 |
| | g/ml | g/ml |
| Bulk Density | 0.75 | 0.75 |
| Tapped Density | 0.88 | 0.88 |
| Hausner Ratio | 1.17 | 1.17 |
| Carr Index | 14.77% | 14.77% |

1. A compressed core for a pharmaceutical dosage form comprising a mixture of (a) at least one pharmaceutically acceptable organic acid, and (b) at least one pharmaceutically acceptable excipient, wherein the pharmaceutically acceptable organic acid is present in an amount of about 50-95% by weight of the core.

2. The compressed core of claim 1 wherein the pharmaceutically acceptable organic acid is present in an amount of about 50-85% by weight of the core.

3. The compressed core of claim 1 wherein the pharmaceutically acceptable organic acid is present in an amount of about 60-90% by weight of the core.

4. The compressed core of claim 1 wherein the pharmaceutically acceptable organic acid is present in an amount of about 85% by weight of the core.

5. The compressed core of claim 1, wherein the pharmaceutically acceptable organic acid has a pKa of about 5.4 or less.

6. The compressed core of claim 1, wherein the pharmaceutically acceptable organic acid has a pKa of about 2.9 to about 5.4.

7. The compressed core of claim 1, wherein the pharmaceutically acceptable organic acid has an aqueous solubility at 20° C. of 4 grams/litre.

8. The compressed core of claim 1, wherein the pharmaceutically acceptable organic acid is selected from the group consisting of fumaric acid, tartaric acid, citric acid, succinic acid, adipic acid, malic acid, maleic acid, lactic acid, or a mixture of one or more thereof.

9. The compressed core of claim 1, wherein the pharmaceutically acceptable organic acid is selected from the group consisting of fumaric acid, tartaric acid, citric acid, and lactic acid or a mixture of one or more thereof.

10. The compressed core of claim 1, wherein the pharmaceutically acceptable organic acid is tartaric acid.

11. The compressed core of claim 1, wherein the at least one pharmaceutically acceptable excipient is selected from the group consisting of a filler, binder, diluent, and lubricant or mixtures thereof.

12. The compressed core of claim 1, wherein the at least one pharmaceutically acceptable excipient is selected from the group consisting of microcrystalline cellulose, lactose, sorbitol, dextrose, sucrose, mannitol, dibasic calcium phosphate, starch, and mixtures thereof, including mixtures of starch and lactose.

13. The compressed core of claim 1, wherein the pharmaceutically acceptable excipient is microcrystalline cellulose.

14. The compressed core of claim 11, wherein the lubricant is selected from the group consisting of sodium stearyl fumarate, stearic acid, magnesium stearate, calcium stearate, zinc stearate, talc and glyceryl behenate.

15. The compressed core of claim 11, wherein the lubricant is magnesium stearate.

16. The compressed core of claim 1, wherein the pharmaceutically acceptable excipient component (b) is present in an amount of about 5-50%, by weight of the core.

17. The compressed core of claim 1, wherein the pharmaceutically acceptable excipient component (b) is present in an amount of about 15-50% by weight of the core.

18. The compressed core of claim 1, wherein the pharmaceutically acceptable excipient component (b) is present in an amount of about 10-40% by weight of the core.

19. The compressed core of claim 1, wherein the pharmaceutically acceptable excipient component (b) is present in an amount of about 15% by weight of the core.

20. The compressed core of claim 1, consisting essentially of a mixture of (a) about 50-95% by weight of a pharmaceutically acceptable organic acid and (b) about 5-50% of at least one pharmaceutically acceptable excipient.

21. The compressed core of claim 20, wherein (a) is present in an amount of about 60-95% by weight, and (b) is present in an amount of about 5-40% by weight.

22. The compressed core of claim 20, wherein (a) is present in an amount of about 70-95% by weight, and (b) is present in an amount of about 5-30% by weight.

23. The compressed core of claim 20, wherein (a) is present in an amount of about 80-90% by weight, and (b) is present in an amount of about 10-20% by weight.

24. The compressed core of claim 11, wherein a lubricant is present in an amount of about 0.05 to about 2 wt % relative to the weight of the core.

25. The compressed core of claim 1, wherein the core is prepared by direct compression of a mixture comprising components (a) and (b).

26. The compressed core of claim 25, wherein the compression is carried out without the addition of a liquid or solvent.

27. The compressed core of claim 1, wherein the friability of the core is 0.1% or less.

28. The compressed core of claim 1, wherein the friability of the core is about 0.1%-0.02%.

29. The compressed core of claim 1, wherein the cores have a diameter of about 3 mm or less.

30. The compressed core of claim 1, wherein the cores have a diameter of about 2 mm or less.

31. The compressed core of any of claim 29, wherein the cores have a diameter of at least about 1.6 mm.

32. The compressed core of claim 1, wherein the cores have a diameter of about 1.7 to about 2.5 mm.

33. The compressed core of claim 1, wherein the cores have a diameter of about 1.7 to about 2.0 mm.

34. The compressed core of claim 1, wherein the cores have a diameter of about 1.8 mm.

35. A pharmaceutical composition comprising a compressed core according to claim 1, wherein the core is coated with a drug layer comprising a drug having a pH dependent solubility profile, wherein the solubility is greater at acidic pH (i.e. pH<7), and at least one pharmaceutically acceptable excipient.

36. A pharmaceutical composition according to claim 35, wherein the pharmaceutically acceptable excipient is selected from the group consisting of a binder, diluent, plasticizer and an anti-tacking (anti-adherant) agent, and mixtures thereof.

37. A pharmaceutical composition according to claim 35, wherein the drug layer contains a binder or a mixture of binders, a plasticizer, and an anti-tacking (anti-adherant) agent.

38. A pharmaceutical composition according to claim 35, wherein the drug layer contains a combination of binder and an anti-tacking agent.

39. A pharmaceutical composition according to claim 36, wherein the binder is selected from the group consisting of cellulosic polymers such as hydroxypropylmethyl cellulose, hydroxypropyl cellulose, hydroxyethyl cellulose, ethyl cellulose, gelatin, methyl cellulose, pregelatinized starch, acacia, alginic acid, sodium carboxymethyl cellulose gum arabic, polyvinyl pyrrolidone, polyvinyl alcohol, and copolymers of N-vinyl pyrrolidine and vinyl acetate or mixtures thereof.

40. A pharmaceutical composition according to claim 36, wherein the binder is selected from the group consisting of hydroxypropylmethyl cellulose and hydroxypropyl cellulose (e.g. Klucel LF) or mixtures thereof.

41. A pharmaceutical composition according to claim 36, wherein the binder is hydroxypropyl cellulose.

42. A pharmaceutical composition according to claim 36, wherein the binder in the drug layer is present in a concentration of about 5 to about 30 wt % relative to the weight of the drug layer.

43. A pharmaceutical composition according to claim 36, wherein the weight ratio of drug to binder in the drug layer is from about 10:1 to about 1:1.

44. A pharmaceutical composition according to claim 36, wherein the weight ratio of drug to binder in the drug layer is from about 6:1 to about 4:1.

45. A pharmaceutical composition according to claim 36, wherein the plasticizer is selected from the group consisting of polyethylene glycol (preferably polyethylene glycol 400), triethyl citrate, tributyl citrate, glycerin, dibutyl sebacate, triacetin and diethylphthalate, or mixtures thereof.

46. A pharmaceutical composition according to claim 36, wherein the plasticiser is present in the drug layer in a concentration of about 2 to about 25 wt % relative to the weight of the drug layer.

47. A pharmaceutical composition according to claim 36, wherein the anti-tacking agent is selected from the group consisting of magnesium carbonate, titanium dioxide, microcrystalline cellulose, polyethylene glycol, colloidal silica, corn starch and talc, or mixtures thereof.

48. A pharmaceutical composition according to claim 36, wherein the anti-tacking agent is present in an concentration range of about 5 wt % to about 25 wt % relative to the weight of the drug layer.

49. A pharmaceutical composition according to claim 35, wherein the compressed core and the drug layer are separated by a subcoat layer.

50. A pharmaceutical composition according to claim 49, wherein the subcoat layer comprises at least one pharmaceutically acceptable excipient selected from one or more of the group consisting of binder, anti-tacking agent, surfactant (emulsifier), and plasticizer.

51. A pharmaceutical composition according to claim 50, wherein the binder in the subcoat layer is selected the group consisting of cellulosic polymers such as hydroxypropylmethyl cellulose, hydroxypropyl cellulose, hydroxyethyl cellulose, ethyl cellulose, gelatin, methyl cellulose, pregelatinized starch, acacia, alginic acid, sodium carboxymethyl cellulose gum arabic, polyvinyl pyrrolidone, polyvinyl alcohol, and copolymers of N-vinyl pyrrolidine and vinyl acetate, or a mixture thereof.

52. A pharmaceutical composition according to claim 50, wherein the binder is selected from hydroxypropylmethyl cellulose and ethyl cellulose or a combination thereof.

53. A pharmaceutical composition according to claim 50, wherein the binder is present in the subcoat layer in a concentration of about 20 to about 95 wt % relative to the weight of the subcoat layer.

54. A pharmaceutical composition according to claim 50, wherein the anti-tacking agent is selected from the group consisting of magnesium carbonate, titanium dioxide, microcrystalline cellulose, polyethylene glycol, colloidal silica, corn starch and talc and mixtures thereof.

55. A pharmaceutical composition according to claim 50, wherein the plasticizer is selected from the group consisting of polyethylene glycol (particularly polyethylene glycol 400), triethyl citrate, tributyl citrate, glycerin, dibutyl sebacate, triacetin and diethylphthalate or a combination thereof.

56. A pharmaceutical composition according to claim 50, wherein the plasticizer is present in a concentration of about 5 to about 30 wt % relative to the weight of the subcoat.

57. A pharmaceutical composition according to claim 50, wherein the surfactant or emulsifier is selected from the group consisting of benzalkonium chloride, cetyl alcohol, polysorbate 80, sodium lauryl sulfate and sorbitan esters including sorbitan mono-palmitate, or mixtures thereof.

58. A compressed core according to claim 1 or a pharmaceutical composition according to claim 35 further comprising a dissolution enhancer, said dissolution enhancer preferably being a pore former, osmotic agent, disintegrant or surfactant.

59. A pharmaceutical composition according to claim 58, where in the pore former is present in an amount of about 5-20% w/w of the layer or core it is present in.

60. A pharmaceutical composition according to claim 58 wherein the dissolution enhancer is present in the drug layer.

61. A pharmaceutical composition according to claim 58, wherein the dissolution enhancer is a pore former.

62. A pharmaceutical composition according to claim 33, wherein the drug layer is coated with a protective top coat, an extended-release coat or a delayed-release coat.

63. A pharmaceutical composition according to claim 62, wherein the extended-release coat comprises an extended-release polymer.

64. A pharmaceutical composition according to claim 63, wherein the extended-release polymer is selected from the group consisting of ethyl cellulose (e.g. ethylcellulose having a viscosity of about 4 to about 10 cPs, preferably about 5 to about 9 cPs, and more preferably about 7 cPs), hydroxypropyl methylcellulose (HPMC), polyvinyl alcohol (PVA; vinyl alcohol polymer), polymethacrylates, ethyl acrylate-methyl methacrylate copolymers (such as Eudragit RS), hydroxypropyl cellulose (HPC) or a mixture thereof.

65. A pharmaceutical composition according to claim 62, wherein the extended-release layer further comprises a binder or a plasticizer or a mixture thereof.

66. A pharmaceutical composition according to claim 35, wherein the drug is selected from the group consisting of dabigatran, dabigatran prodrugs such as dabigatran etexilate, dipyridamole, aliskiren, fingolimod, and retigabine, or prodrugs thereof, and pharmaceutically acceptable salts of the drugs or prodrugs.

67. A multiparticulate dosage form, comprising a plurality of coated cores as defined in claim 35.

68. A multiparticulate dosage form according to claim 67, wherein the multiparticulate dosage form comprises a plurality of pharmaceutical compositions as defined in claim 35 wherein the compositions are filled into capsules.

69. A multiparticulate dosage form according to claim 67, wherein the drug is dabigatran etexilate, preferably dabigatran etexilate mesylate.

70. A multiparticulate dosage form according to claim 67 wherein the dosage form provides from 25 mg to 300 mg of dabigatran etexilate.

71. A process for the preparation of the compressed core of claim 1, comprising:
(i) admixing the pharmaceutically acceptable acid with the at least one pharmaceutically acceptable excipient to form a mixture, and
(ii) direct compression of the mixture.

72. A process according to claim 71, wherein the final blend for direct compression is prepared without the addition of a liquid or solvent.

73. A process for preparing the pharmaceutical composition according to claim 35 comprising:
(i) preparing a compressed core by the process of claim 71, and
(ii) optionally applying a sub-coat layer over the compressed core,
(iii) applying a drug layer over the compressed core or sub-coated compressed core, and
(iv) optionally applying a protective top coat, an extended release coat or a delayed release coat over the drug layer.

74. A process according to claim 73, wherein the composition or a plurality thereof is filled into a capsule.

75. A process according to claim 74, wherein the capsule provides a 75 mg, 110 mg, 150 mg or 220 mg dose of the drug.
